(19) United States
(12) Patent Application Publication
(10) Pub. No.: US 2026/0191761 A1
(43) Pub. Date: Jul. 9, 2026

(54) COSMETIC COMPOSITION

(71) Applicant: Shiseido Company, Ltd., Chuo-ku, Tokyo (JP)

(72) Inventors: Takashi OKA, Tokyo (JP); Hiroko MANABE, Tokyo (JP); Yuji ITO, Tokyo (JP)

(73) Assignee: Shiseido Company, Ltd., Chuo-ku, Tokyo (JP)

(21) Appl. No.: 19/133,401

(22) PCT Filed: Dec. 8, 2023
(86) PCT No.: PCT/JP2023/043987

§ 371 Date: May 28, 2025

(30) Foreign Application Priority Data
Dec. 28, 2022 (JP) 2022-212226

(51) Int. Cl. A61K 8/86; A61K 8/37; A61Q 17/04

(52) U.S. Cl. CPC A61K 8/86; A61K 8/375; A61Q 17/04; A61K 2800/592

(57) ABSTRACT

Provided is a novel percutaneous penetration inhibitor for an oily component such as an ultraviolet absorber and/or preservative having a log P value of 2.0 or greater and the novel percutaneous penetration inhibitor.

A composition that is a cosmetic composition comprising a polymer having a structure represented by formula (1) below and an oily component,

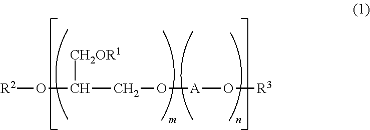

where in the formula (1), R¹, R², and R³ are each independently a hydrogen atom and an alkyl group having 1 to 4 carbon atoms; A is an alkylene group having 2 to 4 carbon atoms; and m and n are each independently 1.0 to 50, and wherein a number average molecular weight of the polymer is 10,000 or less, an IOB value of the polymer is 0.4 to 1.8, and the oily component is an ultraviolet absorber and/or preservative having a log P value of 2.0 or greater.

### FIELD

The present invention relates to a cosmetic composition.

### BACKGROUND

Oily components such as ultraviolet absorbers and preservatives are blended into various cosmetics. Such oily components are preferably retained on the skin surface as much as possible without penetrating into the subcutaneous layer.

For example, PTL 1 discloses a cosmetic that can inhibit percutaneous penetration of a specific ultraviolet absorber (for example, octyl methoxycinnamate). More specifically, the cosmetic of PTL 1 is a cosmetic containing octyl methoxycinnamate and polypropylene glycol having a number average molecular weight of 500 to 2500 (excluding cleansing cosmetics containing polypropylene glycol butyl ether).

### CITATION LIST

### Patent Literature

[PTL 1] Japanese Unexamined Patent Publication (Kokai) No. 2002-284622

### SUMMARY

### Technical Problem

However, there remains room for improvement in terms of inhibiting percutaneous penetration of not only octyl methoxycinnamate but also other ultraviolet absorbers and preservatives.

The present invention aims to improve the above circumstances, and an object thereof is to provide a novel percutaneous penetration inhibitor for an oily component such as an ultraviolet absorber and/or preservative having a log P value of 2.0 or greater and a cosmetic composition comprising the novel percutaneous penetration inhibitor.

### Solution to Problem

The present invention that achieves the above object is as follows.

### <Aspect 1>

A composition that is a cosmetic composition comprising a polymer having a structure represented by formula (1) below and an oily component,

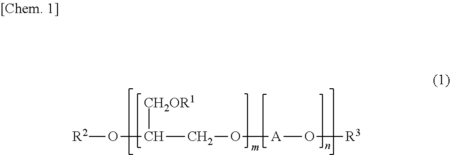

where in the formula (1), R¹, R², and R³ are each independently a hydrogen atom and an alkyl group having 1 to 4 carbon atoms; A is an alkylene group having 2 to 4 carbon atoms; and m and n are each independently 1.0 to 50, and
wherein a number average molecular weight of the polymer is 10,000 or less,
wherein an IOB value of the polymer is 0.4 to 1.8, and
wherein the oily component is an ultraviolet absorber and/or preservative having a log P value of 2.0 or greater.

### <Aspect 2>

The composition according to Aspect 1, wherein
in the formula (1), A is represented by formula (2) below:

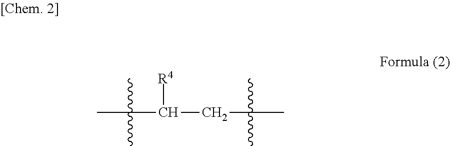

where in the formula (2), R⁴ is an alkyl group having 1 or 2 carbon atoms.

### <Aspect 3>

The composition according to Aspect 1 or 2, wherein the polymer is a random copolymer.

### <Aspect 4>

The composition according to any one of Aspects 1 to 3, wherein in the formula (1), m is 2 or greater and at least a portion of R¹ includes a methyl group.

### <Aspect 5>

The composition according to any one of Aspects 1 to 4, wherein in the formula (1), at least one of R² and R³ is a methyl group.

### <Aspect 6>

The composition according to any one of Aspects 1 to 5, wherein the oily component has a log P value of 15.0 or less.

### <Aspect 7>

The composition according to any one of Aspects 1 to 6, wherein the polymer is a percutaneous penetration inhibitor of the oily component.

### <Aspect 8>

Use of a polymer having a structure represented by formula (1) below, as a percutaneous penetration inhibitor of an oily component that is an ultraviolet absorber and/or preservative having a log P value of 2.0 or greater:

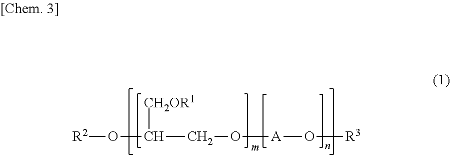

where in the formula (1), R¹, R², and R⁵ are each independently a hydrogen atom and an alkyl group having 1 to 4 carbon atoms; A is an alkylene group having 2 to 4 carbon atoms; and m and n are each independently 1.0 to 50, and wherein
a number average molecular weight of the polymer is 10,000 or less, and
an IOB value of the polymer is 0.4 to 1.8.

### <Aspect 9>

The use according to Aspect 8, wherein in the formula (1), A is represented by formula (2) below:

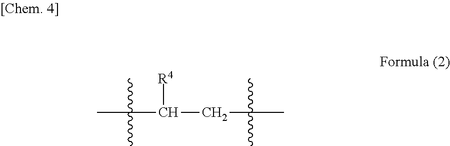

where in the formula (2), R⁴ is an alkyl group having 1 or 2 carbon atoms.

### <Aspect 10>

The use according to Aspect 8 or 9, wherein the polymer is a random copolymer.

### <Aspect 11>

The use according to any one of Aspects 8 to 10, wherein in the formula (1), m is 2 or greater and at least a portion of R¹ includes a methyl group.

### <Aspect 12>

The use according to any one of Aspects 8 to 11, wherein in the formula (1), at least one of R² and R³ is a methyl group.

### <Aspect 13>

The use according to any one of Aspects 8 to 12, wherein the oily component has a log P value of 15.0 or less.

### Advantageous Effects of Invention

According to the present invention, a novel percutaneous penetration inhibitor for an oily component such as an ultraviolet absorber and/or preservative having a log P value of 2.0 or greater and a cosmetic composition comprising the novel percutaneous penetration inhibitor can be provided.

### BRIEF DESCRIPTION OF DRAWINGS

FIG. 1 is a chart showing stratum corneum sorption amounts (stratum corneum separated by layer) of composition samples of Example 1, Example 2, and Comparative Example 1.

FIG. 2 is a chart showing stratum corneum sorption amounts (average of all layers of stratum corneum) of composition samples of Example 1, Example 2, and Comparative Example 1.

FIG. 3 is a chart showing stratum corneum sorption amounts (stratum corneum separated by layer) of composition samples of Example 3 and Comparative Example 2.

FIG. 4 is a chart showing stratum corneum sorption amounts (average of all layers of stratum corneum) of composition samples of Example 3 and Comparative Example 2.

FIG. 5 is a chart showing stratum corneum sorption amounts (stratum corneum separated by layer) of composition samples of Example 4 and Comparative Example 3.

FIG. 6 is a chart showing stratum corneum sorption amounts (average of all layers of stratum corneum) of composition samples of Example 4 and Comparative Example 3.

### DESCRIPTION OF EMBODIMENTS

Hereinafter, embodiments of the present invention will be described in detail. Note that the present invention is not limited to the following embodiments, and various modifications can be made within the scope of the invention.

### <<Cosmetic Composition>>

The cosmetic composition of the present invention (hereinafter, also simply referred to as “composition of the present invention”) is a composition that is in
a cosmetic composition comprising a polymer having a structure represented by formula (1) below and an oily component,

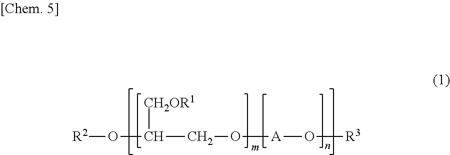

where in the formula (1), R¹, R², and R³ are each independently a hydrogen atom and an alkyl group having 1 to 4 carbon atoms; A is an alkylene group having 2 to 4 carbon atoms; and m and n are each independently 1.0 to 50, and
wherein a number average molecular weight of the polymer is 10,000 or less,
wherein an IOB value of the polymer is 0.4 to 1.8, and
wherein the oily component is an ultraviolet absorber and/or preservative having a log P value of 2.0 or greater.

The composition of the present invention comprises the polymer of the present invention described below. The polymer of the present invention has an effect of inhibiting penetration of a specific oily component (i.e., oily component that is an ultraviolet absorber and/or preservative having a log P value of 2.0 or greater) into the skin. Therefore, it can be said that the polymer of the present invention is a percutaneous penetration inhibitor of such a specific oil component. In other words, the polymer of the present invention can be used as a percutaneous penetration inhibitor of such a specific oily component contained in the composition of the present invention.

Without wishing to be bound by theory, it is presumed that this is derived from the characteristics of the polymer of the present invention, i.e., the unique structure indicated by the formula (1), number average molecular weight in a specific range (10,000 or less), and IOB (inorganic organic balance) value in a specific range (0.4 to 1.8).

The polymer of the present invention is considered amphiphilic from the unique structure thereof. It is presumed that the polymer of the present invention, having this amphiphilic property, demonstrates an effect of retaining a specific oily component (i.e., oily component that is an ultraviolet absorber and/or preservative having a log P value of 2.0 or greater) to retain the oily component on the surface of the skin. In other words, it is presumed that by using the polymer of the present invention in combination with a specific oily component, the penetration thereof into the skin is decreased.

### <Polymer of Present Invention>

The polymer of the present invention has a structure represented by formula (1) below:

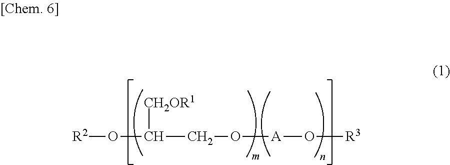

In the formula (1), R¹, R², and R³ are each independently a hydrogen atom or an alkyl group having 1 to 4 carbon atoms. In the present invention, examples of the alkyl group having 1 to 4 carbon atoms include a methyl group, an ethyl group, an n-propyl group, an isopropyl group, an n-butyl group, a sec-butyl group, and a tert-butyl group. These alkyl groups may further comprise a substituent as long as the effect of the present invention is not impaired. The substituent is not particularly limited, and examples include halogeno groups.

According to one embodiment of the present invention, in the formula (1), R¹, R², and R³ may each independently be a hydrogen atom, a methyl group, an ethyl group, an n-propyl group, an isopropyl group, an n-butyl group, a sec-butyl group, or a tert-butyl group.

According to one embodiment of the present invention, in the formula (1), R¹ is preferably a hydrogen atom, a methyl group, or an ethyl group. When m is 2 or greater, i.e., when a plurality of R¹ are present, each R¹ may be the same or different, and in particular, at least a portion of R¹ preferably includes a methyl group. The case where at least a portion of R¹ includes a methyl group is preferable, for example, from the viewpoint of improving feeling of use of the composition of the present invention.

According to one embodiment of the present invention, in the formula (1), R¹ may be in a state where a hydrogen atom and an alkyl group having 1 to 4 carbon atoms coexist. Specifically, one portion of R¹ may include a hydrogen atom, and the other portion thereof may include an alkyl group having 1 to 4 carbon atoms. Preferably, one portion of R¹ includes a hydrogen atom, and the other portion thereof includes a methyl group.

According to one embodiment of the present invention, in the formula (1), R² is preferably a hydrogen atom, a methyl group, or an ethyl group, and particularly, is more preferably a methyl group.

According to one embodiment of the present invention, in the formula (1), R² may be in a state where a hydrogen atom and an alkyl group having 1 to 4 carbon atoms coexist. Specifically, one portion of R² may include a hydrogen atom, and the other portion thereof may include an alkyl group having 1 to 4 carbon atoms. Preferably, one portion of R-includes a hydrogen atom, and the other portion thereof includes a methyl group.

According to one embodiment of the present invention, in the formula (1), R³ is preferably a hydrogen atom, a methyl group, or an ethyl group, and particularly, is more preferably a methyl group.

According to one embodiment of the present invention, in the formula (1), at least one of R² and R³ is preferably a methyl group.

In the formula (1), A is an alkylene group having 2 to 4 carbon atoms. More specifically, A may be, for example, an ethylene group, a propylene group, a butylene group, or an isobutylene group, but is not limited thereto. In particular, A is preferably represented by formula (2) below:

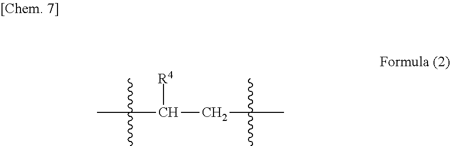

In the formula (2), R⁴ is an alkyl group having 1 or 2 carbon atoms. More specifically, R⁴ may be a methyl group or an ethyl group.

In the formula (1), m and n represent an average number of moles added for each structural unit. The m and n are each independently 1.0 to 50, and more specifically, may be 1.0 or greater, 1.5 or greater, 2.0 or greater, 3.0 or greater, 4.0 or greater, 5.0 or greater, 6.0 or greater, 7.0 or greater, 8.0 or greater, 9.0 or greater, or 10 or greater, and may be 50 or less, 45 or less, 40 or less, 35 or less, 34 or less, 32 or less, 30 or less, 28 or less, 26 or less, 25 or less, 24 or less, 22 or less, 20 or less, 18 or less, 16 or less, 15 or less, 14 or less, 12 or less, 10 or less, or 5.0 or less.

In the formula (1), m is preferably 1.0 or greater and 14 or less, and more preferably 2.0 or greater and 5.0 or less. In addition, in the formula (1), n is preferably 2.0 or greater and 34 or less, and more preferably 6.0 or greater and 12 or less.

According to one embodiment of the present invention, in the formula (1), m+n may be 4.0 or greater, 4.5 or greater, 5.0 or greater, 6.0 or greater, 7.0 or greater, 8.0 or greater, 9.0 or greater, 10 or greater, 11 or greater, 12 or greater, 13 or greater, 14 or greater, or 15 or greater, and may be 50 or less, 45 or less, 40 or less, 35 or less, 30 or less, 25 or less, 20 or less, 15 or less, or 12 or less.

According to one embodiment of the present invention, in the formula (1), min may be 1:10 to 10:1.

For the polymer of the present invention, the number average molecular weight is 10,000 or less. More specifically, the number average molecular weight of the polymer of the present invention, for example, may be 10,000 or less, 5,000 or less, 3,500 or less, 3,200 or less, 3,000 or less, 2,800 or less, 2,500 or less, 2,200 or less, 2,000 or less, 1,800 or less, 1,500 or less, 1,400 or less, 1,300 or less, 1,200 or less, 1,100 or less, 1,000 or less, 900 or less, 800 or less, 700 or less, or 600 or less, and may be 130 or greater, 150 or greater, 200 or greater, 250 or greater, 300 or greater, 350 or greater, 400 or greater, 450 or greater, 500 or greater, 550 or greater, 600 or greater, 650 or greater, 700 or greater, 750 or greater, 800 or greater, 850 or greater, 900 or greater, 950 or greater, or 1,000 or greater. In addition, the number average molecular weight of the polymer of the present invention is preferably 300 or greater and 3,500 or less, and more preferably 500 or greater and 1,200 or less.

The IOB value of the polymer of the present invention is 0.4 to 1.8. More specifically, the IOB value of the polymer of the present invention, for example, may be 0.4 or greater, 0.5 or greater, 0.6 or greater, 0.7 or greater, 0.8 or greater, 0.9 or greater, or 1.0 or greater, and may be 1.8 or less, 1.6 or less, 1.4 or less, or 1.2 or less. In addition, the IOB value of the polymer of the present invention is preferably 0.7 or greater and 1.2 or less.

The IOB value is an abbreviation for inorganic/organic balance, a value that represents a ratio of inorganic value to organic value, and acts as an indicator showing the degree of polarity of an organic compound. The IOB value, specifically, is represented as IOB value=inorganic value/organic value. Regarding each of the “inorganic value” and “organic value”, “inorganic value” and “organic value” are set according to various atoms or functional groups, such that, for example, the “organic value” for one carbon atom in a molecule is 20, and the “inorganic value” for one hydroxyl group is 100. The IOB value of an organic compound can be calculated by summing the “inorganic values” and “organic values” of all atoms and functional groups in the organic compound (refer to, for example, Yoshio Koda, “Organic Conceptual Diagrams-Fundamentals and Applications-”, pp. 11 to 17, Sankyo Publishing Co., Ltd., 1984). Note that regarding the method of determining IOB values, the method described in the Examples can be referenced.

The polymer of the present invention may be a block copolymer, or may be a random copolymer, but is preferably a random copolymer.

The manufacturing method for the polymer of the present invention is not particularly limited and, for example, may be carried out by addition polymerization of epoxides corresponding to each structural unit. The addition polymerization may be block polymerization, or may be random polymerization, but is preferably random polymerization.

The manufacturing method for the polymer of the present invention may further comprise further reacting an alkyl halide with the polymer obtained by the above addition polymerization. A hydroxyl group in a molecule of the polymer obtained via the reaction with an alkyl halide is blocked by the alkyl group, and as a result, hydrophobicity of the entire polymer can be adjusted as desired. In the present invention, blocking rate of hydroxyl group by alkyl group within a molecule of the polymer, for example, may be 30% or greater, 35% or greater, 40% or greater, 45% or greater, 50% or greater, or 55% or greater, and may be 60% or less, 59% or less, 58% or less, 57% or less, 56% or less, or 55% or less. Note that the blocking rate of hydroxyl group by alkyl group can be determined by the method described in the Examples.

The polymer of the present invention, as one of the characteristics thereof, has both high water solubility and high lipid solubility.

The degree of solubility in water of the polymer of the present invention can be, for example, 10% by mass or greater, 15% by mass or greater, 20% by mass or greater, 25% by mass or greater, 30% by mass or greater, 35% by mass or greater, 40% by mass or greater, 45% by mass or greater, or 50% by mass or greater.

As an indicator of lipid solubility of the polymer of the present invention, for example, degree of solubility in olive oil can be used. The degree of solubility in olive oil of the polymer of the present invention, for example, can be 0.01% by mass or greater, 0.05% by mass or greater, 0.10% by mass or greater, 0.50% by mass or greater, or 1.00% by mass or greater. Note that the upper limit value of the degree of solubility in olive oil of the polymer of the present invention is not particularly limited and, for example, may be 10% by mass or less.

In the composition of the present invention, the content of the polymer of the present invention is not particularly limited and, for example, may be 0.001% by mass or greater, 0.005% by mass or greater, 0.01% by mass or greater, 0.05% by mass or greater, 0.1% by mass or greater, 0.5% by mass or greater, 1.0% by mass or greater, 2.0% by mass or greater, 3.0% by mass or greater, 4.0% by mass or greater, or 5.0% by mass or greater, and may be 90% by mass or less, 80% by mass or less, 70% by mass or less, 60% by mass or less, 50% by mass or less, 40% by mass or less, 30% by mass or less, 20% by mass or less, 10% by mass or less, 8.0% by mass or less, 5.0% by mass or less, 3.0% by mass or less, or 1.0% by mass or less.

The polymer of the present invention, when used as a percutaneous penetration inhibitor of an oily component that is an ultraviolent absorber and/or preservative having a log P value of 2.0 or greater, for example, may be 1.0 parts by mass or more, 5.0 parts by mass or more, 10 parts by mass or more, 20 parts by mass or more, 30 parts by mass or more, 40 parts by mass or more, 50 parts by mass or more, 60 parts by mass or more, 70 parts by mass or more, 80 parts by mass or more, 90 parts by mass or more, or 100 parts by mass or more, and may be 10000 parts by mass or less, 5000 parts by mass or less, 1000 parts by mass or less, 500 parts by mass or less, or 100 parts by mass or less, relative to a total of 100 parts by mass of the oily component that is an ultraviolent absorber and/or preservative having a log P value of 2.0 or greater.

<Oily Component that is Ultraviolet Absorber and/or Preservative Having Log P Value of 2.0 or Greater>

The composition of the present invention comprises a specific oily component. The specific oily component according to the present invention is an ultraviolet absorber and/or preservative having a log P value of 2.0 or greater. Note that in the present invention, the term “preservative” also includes the commonly used “antioxidant”.

An “oily component that is an ultraviolet absorber and/or preservative having a log P value of 2.0 or greater” herein refers to an ultraviolet absorber having a log P value of 2.0 or greater and/or a preservative having a log P value of 2.0 or greater.

A “log P value” represents a ratio of equilibrium concentrations of a substance dissolved in two phases, octanol and water, and is an indicator showing the hydrophilicity or hydrophobicity of the component (nonionic substance). In particular, it is known that when the log P value is near 2.0, skin penetration of the component (nonionic substance) is maximized, and when the log P value is less than that, skin penetration of the component (nonionic substance) decreases (for example, “Non-formulation Parameters That Affect Penetrant-Skin-Vehicle Interactions and Percutaneous Absorption”, JE Grice et al., “Percutaneous Penetration Enhancers Drug Penetration Into/Through the Skin” (2017), pp. 45 to 75).

The log P value can be obtained by referencing a database in which log P values of many compounds are listed, available from, for example, Daylight Chemical Information Systems, Inc. (Daylight CIS) or PubChem (National Center for Biotechnology Information). If no actual log P value is available, the value can be calculated with the program “CLOGP” (Daylight CIS).

### (Ultraviolet Absorber Having Log P Value of 2.0 or Greater)

In the present invention, the log P value of the ultraviolet absorber should be 2.0 or greater and, for example, may be 2.1 or greater, 2.2 or greater, 2.3 or greater, 2.4 or greater, 2.5 or greater, 2.6 or greater, 2.7 or greater, 2.8 or greater, 2.9 or greater, 3.0 or greater, 3.1 or greater, 3.2 or greater, 3.3 or greater, 3.4 or greater, 3.5 or greater, 3.6 or greater, 3.7 or greater, 3.8 or greater, 3.9 or greater, 4.0 or greater, 4.1 or greater, 4.2 or greater, 4.3 or greater, 4.4 or greater, 4.5 or greater, 4.6 or greater, 4.7 or greater, 4.8 or greater, 4.9 or greater, 5.0 or greater, 5.1 or greater, 5.2 or greater, 5.3 or greater, 5.4 or greater, 5.5 or greater, 5.6 or greater, 5.7 or greater, 5.8 or greater, 5.9 or greater, 6.0 or greater, 6.1 or greater, 6.2 or greater, 6.3 or greater, 6.4 or greater, 6.5 or greater, 6.6 or greater, 6.7 or greater, 6.8 or greater, 6.9 or greater, or 7.0 or greater. The upper limit value of the log P value of the ultraviolet absorber is not particularly limited and, for example, may be 15.0 or less, 14.0 or less, 13.0 or less, 12.0 or less, 11.0 or less, 10.0 or less, 9.0 or less, 8.0 or less, 7.0 or less, 6.0 or less, or 5.0 or less.

In the present invention, specific examples of the ultraviolet absorber having a log P value of 2.0 or greater are indicated below, but are not limited thereto. Specifically, examples of the ultraviolet absorber having a log P value of 2.0 or greater include octyl methoxycinnamate (log P value: 5.7) and 4-tert-butyl-4′-methoxydibenzoylmethane (log P value: 4.8).

In the composition of the present invention, the content of the ultraviolet absorber having a log P value of 2.0 or greater is not particularly limited and, for example, may be 0.001% by mass or greater, 0.005% by mass or greater, 0.01% by mass or greater, 0.05% by mass or greater, 0.1% or greater, 0.5% by mass or greater, 1.0% by mass or greater, 1.5% by mass or greater, 2.0% by mass or greater, 2.5% by mass or greater, 3.0% by mass or greater, 3.5% by mass or greater, 4.0% by mass or greater, 4.5% by mass or greater, 5.0% by mass or greater, 5.5% by mass or greater, 6.0% by mass or greater, 6.5% by mass or greater, 7.0% by mass or greater, or 7.5% by mass or greater, and may be 20% by mass or less, 15% by mass or less, 10% by mass or less, or 8.0% by mass or less.

### (Preservative Having Log P Value of 2.0 or Greater)

In the present invention, the log P value of the preservative should be 2.0 or greater and, for example, may be 2.1 or greater, 2.2 or greater, 2.3 or greater, 2.4 or greater, 2.5 or greater, 2.6 or greater, 2.7 or greater, 2.8 or greater, 2.9 or greater, 3.0 or greater, 3.1 or greater, 3.2 or greater, 3.3 or greater, 3.4 or greater, 3.5 or greater, 3.6 or greater, 3.7 or greater, 3.8 or greater, 3.9 or greater, 4.0 or greater, 4.1 or greater, 4.2 or greater, 4.3 or greater, 4.4 or greater, 4.5 or greater, 4.6 or greater, 4.7 or greater, 4.8 or greater, 4.9 or greater, 5.0 or greater, 5.1 or greater, 5.2 or greater, 5.3 or greater, 5.4 or greater, 5.5 or greater, 5.6 or greater, 5.7 or greater, 5.8 or greater, 5.9 or greater, 6.0 or greater, 6.1 or greater, 6.2 or greater, 6.3 or greater, 6.4 or greater, 6.5 or greater, 6.6 or greater, 6.7 or greater, 6.8 or greater, 6.9 or greater, or 7.0 or greater. The upper limit value of the log P value of the preservative is not particularly limited and, for example, may be 15.0 or less, 14.0 or less, 13.0 or less, 12.0 or less, 11.0 or less, 10.0 or less, 9.0 or less, 8.0 or less, 7.0 or less, 6.0 or less, or 5.0 or less.

In the present invention, specific examples of the preservative having a log P value of 2.0 or greater are indicated below, but are not limited thereto. Specifically, examples of the preservative having a log P value of 2.0 or greater include vitamin C palmitate (log P value: 14.0), vitamin E acetate (log P value: 10.8), butylparaben (log P value: 3.6), resveratrol (log P value: 3.1), ethylparaben (log P value: 2.5), and methylparaben (log P value: 2.0).

In the composition of the present invention, the content of the preservative having a log P value of 2.0 or greater is not particularly limited and, for example, may be 0.001% by mass or greater, 0.005% by mass or greater, 0.01% by mass or greater, 0.05% by mass or greater, 0.1% by mass or greater, 0.5% by mass or greater, or 1.0% by mass or greater, and may be 5.0% by mass or less, 2.0% by mass or less, 1.0% by mass or less, or 0.5% by mass or less.

### <Additional Components>

The composition of the present invention may further comprise, as additional components, one or more components generally used in external preparations for skin such as cosmetics and pharmaceuticals, in addition to the polymer of the present invention and the oily component that is an ultraviolet absorber and/or preservative having a log P value of 2.0 or greater described above.

Specifically, examples of the additional components include powder components, oily components other than the oily component that is an ultraviolet absorber and/or preservative having a log P value of 2.0 or greater described above, liquid oils and fats, solid oils and fats, waxes, hydrocarbons, higher fatty acids, higher alcohols, esters, silicones, anionic surfactants, cationic surfactants, amphoteric surfactants, nonionic surfactants, water-soluble polymers, thickeners, film-forming agents, ultraviolet absorbers, sequestering agents, lower alcohols, polyhydric alcohols, saccharides, amino acids, organic amines, polymer emulsions, pH adjusters, skin nutrients, vitamins, antioxidant aids, fragrances, and water, but are not limited thereto.

The dosage form of the composition of the present invention is not particularly limited and, for example, may be a solution system, a solubilized system, an emulsion system (water-in-oil type or oil-in-water type), a powder dispersion system, a water-oil two-layer system, a water-oil-powder three-layer system, a gel, a mist, a spray, a mousse, a roll-on, or a stick, or may be a preparation impregnated or applied to a sheet such as a nonwoven fabric.

The product form of the composition of the present invention is not particularly limited and, for example, may be a facial cosmetic such as a toner, an emulsion, a cream, or a pack; a makeup cosmetic such as a foundation, a base, a lipstick, or an eyeshadow; a sunscreen cosmetic (sunscreen agent); a body cosmetic; a fragranced cosmetic; a skin cleanser such as a makeup remover or a body shampoo; a hair cosmetic such as a hair liquid, a hair tonic, a hair conditioner, a shampoo, a rinse, or a hair growth treatment; or an ointment.

### <Application of Polymer of Present Invention>

The present invention also provides a use of the polymer of the present invention described above as a percutaneous penetration inhibitor of the specific oily component (i.e., oily component, such as an ultraviolet absorber and/or preservative, having a log P value of 2.0 or greater) described above.

Regarding the specific oily component (i.e., oily component, such as an ultraviolet absorber and/or preservative, having a log P value of 2.0 or greater) and the blending amount thereof when using the polymer of the present invention described above as a percutaneous penetration inhibitor of the specific oily component, the section “Composition of present invention” described above can be appropriately referenced.

### EXAMPLES

The present invention will be further described in detail with reference to the Examples below. However, the present invention is not limited thereto.

### Synthesis Examples 1 and 2

In each of the following Synthesis Examples 1 and 2, a polymer having a structure represented by formula (3) below was synthesized:

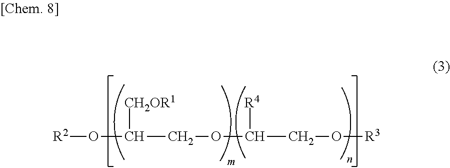

Note that in the formula (3), within the [ ] is a random copolymer.

### Synthesis Example 1

Synthesis Example 1: Polymer 1, where R¹=H; R²=H; R³=CH₃; R⁴=CH₃; m=2.0; and n=6.0

Synthesis was carried out by the method below to obtain Polymer 1.

128 g of methanol and, as a catalyst, 6.4 g of potassium hydroxide were placed inside an autoclave, air inside the autoclave was replaced with dry nitrogen, and the catalyst was completely dissolved at 80° C. while stirring. Next, a mixture of 592 g of glycidol and 1392 g of propylene oxide was added dropwise at 95° C. over 20 h with a dropping apparatus, followed by stirring for 5 h. The reaction composition was taken out from the autoclave, neutralized with hydrochloric acid to a pH of 6 to 7, and treated under reduced pressure of −0.095 MPa (50 mmHg) at 100° C. for 1 h to remove contained moisture. After the treatment, filtration was further carried out to remove any salt generated, and 2000 g of Polymer 1 of Synthesis Example 1 was obtained.

Various physical property values for the obtained Polymer 1 were determined below.

### (Number Average Molecular Weight)

The number average molecular weight of the obtained Polymer 1 was calculated by gel permeation chromatography (GPC) measurement. Using a SHODEX™ GPC101 dedicated GPC system as the system, a SHODEX RI-71s as the differential refractometer, a SHODEX KF-G as the guard column, and three SHODEX KF804L installed in series as columns, tetrahydrofuran as the developing solvent was flowed at a flow rate of 1 ml/min at a column temperature of 40° C. 0.1 ml of a 0.1 wt % tetrahydrofuran solution of the resulting reaction product was injected, and a chromatogram represented by refractive index intensity and elution time was obtained using a BORWIN GPC calculation program. A number average molecular weight was determined from this chromatogram using polyethylene glycol as the standard, and was about 530.

### (Weight Average Molecular Weight)

The weight average molecular weight of the obtained Polymer 1 was determined by GPC calculation in the same manner as above, and was about 859.

### (Polydispersity)

From the results of number average molecular weight and weight average molecular weight determined above, the polydispersity Mw/Mn of Polymer 1 was 1.62.

### (IOB Value)

The IOB value of the obtained Polymer 1 was determined as follows, and was 1.1.

More specifically, in Polymer 1, the organic value was calculated based on 20 for carbon and −10 for an iso-branch. In addition, the inorganic value was calculated based on 100 for a hydroxyl group and 20 for an ether bond.
Methanol site: (1 carbon atom, 1 hydroxyl group)×1
   Organic value: 20
   Inorganic value: 100
Glycidol site: (3 carbon atoms, 1 iso-branch, 1 hydroxyl group, 1 ether bond)×2
   Organic value: (60−10)×2=100
   Inorganic value: (100+20)×2=240
Propylene oxide site: (3 carbon atoms, 1 iso-branch, 1 ether bond)×6
   Organic value: (60−10)×6=300
   Inorganic value: 20×6=120

From the above, the total organic value is 420 and the total inorganic value is 460, and thus the IOB value is 1.09.

### (Cloud Point)

A 5 wt % aqueous solution of the obtained Polymer 1 was prepared, heated to 85° C., and then cooled. The temperature at which a transparent solution was formed was 74° C. Thus, it was found that the cloud point of Polymer 1 was 74° C.

### (Hydroxyl Value)

The hydroxyl value of the obtained Polymer 1 was measured in accordance with the measurement method in JIS K-1557-1, and was 294.

### (Solubility in Water)

Solubility in water of the obtained Polymer 1 was determined by the same method as that for “Compatibility evaluation” described below, and was 50% by mass or greater.

### (Solubility in Olive Oil)

Solubility in olive oil of the obtained Polymer 1 was determined as follows. Specifically, Polymer 1 was mixed with olive oil so as to have a concentration of 1.0 wt %, and appearance was confirmed. “Yes” was indicated if the appearance was uniform. In this case, the solubility in olive oil of Polymer 1 was 1.0% by mass.

Various physical property values of Polymer 1 measured below are shown in Table 1 below.

### Synthesis Example 2

Synthesis Example 2: Polymer 2, where R¹=H and CH₃; R²=H and CH₃; R³=CH₃; R⁴=CH₃; m=2.0; and n=6.0

Synthesis by the method below was carried out to obtain Polymer 2.

128 g of methanol and, as a catalyst, 6.4 g of potassium hydroxide were placed inside an autoclave, air inside the autoclave was replaced with dry nitrogen, and the catalyst was completely dissolved at 80° C. while stirring. Next, a mixture of 592 g of glycidol and 1392 g of propylene oxide was added dropwise at 95° C. over 20 h with a dropping apparatus, followed by stirring for 2 h. Next, 244 g of potassium hydroxide was added, and the inside of the system was replaced with dry nitrogen. After the inside of the system was replaced with dry nitrogen, 200 g of methyl chloride was injected at a temperature of 80 to 130° C. and reacted for 5 h. The reaction composition was then taken out from the autoclave, neutralized with hydrochloric acid to a pH of 6 to 7, and treated under reduced pressure of −0.095 MPa (50 mmHg) at 100° C. for 1 h to remove contained moisture. After the treatment, filtration was further carried out to remove any salt generated, and 1820 g of Polymer 2 was obtained. A sample was taken before reacting the methyl chloride, and the hydroxyl value of the purified polymer was 125. Thus, it was found that the ratio of methyl group to hydrogen atom (CH₃/H) of R² and R³ was 0.57. Specifically, the hydroxyl group blocking rate of Polymer 2 was 57%.

In the same manner as in the case of Polymer 1 mentioned above, various physical property values for the obtained Polymer 2 were determined, and each of the results is shown in Table 1 below.

TABLE 1
(Table 1 Synthesis Example 1 and Synthesis Example 2)

| Synthesis Example No. | | |
|---|---|---|
| (Polymer No.) | 1 | 2 |
| | 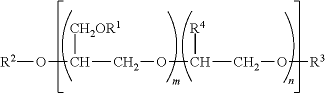 | |
| R¹ | H | CH₃ and H |
| R² | H | CH₃ and H |
| R³ | CH₃ | CH₃ |
| R⁴ | CH₃ | CH₃ |
| Ratio of CH₃ to H in R¹ and R² (CH₃/H) | — | 0.57 |
| m | 2.0 | 2.0 |
| n | 6.0 | 6.0 |
| m + n | 8.0 | 8.0 |
| Polymerization type | Random | Random |
| Number average molecular weight | 530 | 673 |
| Weight average molecular weight | 859 | 956 |
| Polydispersity (Mw/Mn) | 1.62 | 1.42 |
| IOB value | 1.09 | 0.77 |
| Hydroxyl group blocking rate (%) | — | 57 |
| Hydroxyl value (mgKOH/g) | 294 | 125 |
| Cloud point (5% aq.) | 74° C. | 45° C. |
| Solubility (water) | 50% by mass or greater | 30% by mass |
| Solubility (oil) *1 | Yes | Yes |

*1 Synthesized compound was mixed with olive oil so as to have a concentration of 1.0% by mass, and appearance was confirmed. “Yes” was indicated if appearance was uniform.

### <Compatibility Evaluation>

Polymers 1 and 2 synthesized above were each mixed with water and various oils at certain concentrations, stirred, and then left to stand for one day, followed by examining individual compatibility by visual observation. Results thereof are shown in Table 1-2 below. Note that in Table 1-2, “Yes” represents “compatible” and “No” represents “incompatible”.

TABLE 1-2

| | Concentration in each | | |
|---|---|---|---|
| | polymer (% by mass) | Polymer 1 | Polymer 2 |
| Water | 30 | Yes | Yes |
| | 50 | Yes | No |
| Squalane | 1 | No | No |
| Hydrogenated | 1 | No | No |
| polyisobutene | | | |
| Olive oil | 1 | Yes | Yes |
| Cetyl ethylhexanoate | 1 | No | No |
| Macadamia seed oil | 1 | Yes | Yes |
| Isopropyl palmitate | 1 | No | Yes |
| Isopropyl myristate | 1 | No | Yes |
| Ethyl hexyl palmitate | 1 | No | No |

### Examples 1 and 2 and Comparative Example 1

Based on the formulations in Table 2, each of the cosmetic compositions (water-in-oil sunscreens) of the Examples and Comparative Example was prepared by a conventional method. Each of the prepared compositions was subjected to a “human stratum corneum sorption test”. Note that the numerical values in Table 2 refer to blending amounts of the components, and are in units of % by mass.

TABLE 2
(Table 2: Examples 1 and 2 and Comparative Example 1)

| | | | | Compar- |
|---|---|---|---|---|
| | | Example | Example | ative |
| Constituent component | | 1 | 2 | Example 1 |
| Percutaneous | Polymer 1 | 5.0 | — | — |
| penetration | Polymer 2 | — | 5.0 | — |
| inhibitor | | | | |
| Ultraviolet | Octyl | 7.5 | 7.5 | 7.5 |
| absorber | methoxycinnamate*¹ | | | |
| | (logP value: 5.7) | | | |
| Additional | Ion-exchanged water | 42.8 | 42.8 | 47.8 |
| component | Ethanol | 10 | 10 | 10 |
| | Glycerin | 1.0 | 1.0 | 1.0 |
| | Dimethyl distearyl | 1.5 | 1.5 | 1.5 |
| | ammonium hectorite | | | |
| | PEG-9 | 2.0 | 2.0 | 2.0 |
| | polydimethyl- | | | |
| | siloxyethyl | | | |
| | dimethicone | | | |
| | Diisopropyl sebacate | 10 | 10 | 10 |
| | Decamethyltetra- | 20 | 20 | 20 |
| | siloxane | | | |
| | Trisodium edetate | 0.2 | 0.2 | 0.2 |

| | | | |
|---|---|---|---|
| Total | 100 | 100 | 100 |

*¹“Octyl methoxycinnamate”: trade name: Parsol MCX; manufactured by DSM Nutrition Japan K.K.

### <Human Stratum Corneum Sorption Test>

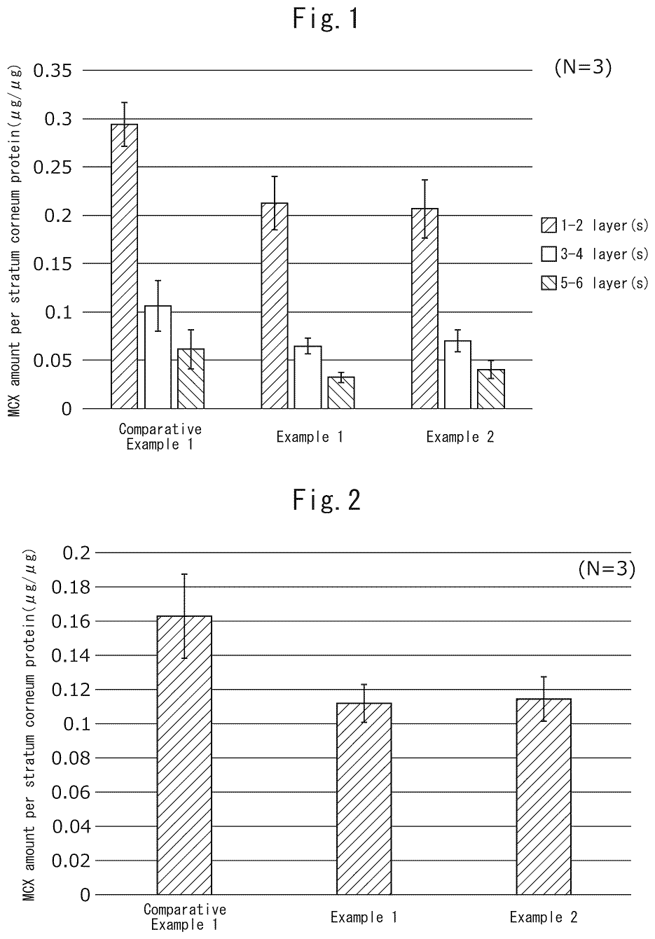

In a testing room, both inner forearms of a subject were washed once with soap. The washed areas were dried by wiping with a towel, and then the subject was left to wait in the testing room for 15 min. A sample of each composition was applied to an inner forearm at a rate of 2 μL/cm². The assignment of application sites for each sample was randomly rotated. 6 h after application of the sample, the application site was washed once with soap to remove the sample adsorbed on the skin surface. The subject was left to wait in the testing room for 15 min, and then the stratum corneum of the site where the sample was applied was collected six times (six layer portions) by tape stripping. After quantifying the protein amount in the stratum corneum collected by each tape, the two pieces of tape for the two layer portions selected in order from the top layer out of the six pieces of tape for the six layer portions were removed. These two pieces of tape were immersed in 2 mL of a solvent (mixed solvent containing water and methanol in a ratio of 1:1) while being subjected to ultrasound emissions for 15 min, and were stored overnight at 37° C. to extract the drug from the tapes. Extracts were collected from the stored liquids, drug amounts were quantified using an Agilent G6120 HPLC system provided with an LC-MS detector, and the stratum corneum sorption amounts per two layer portions of stratum corneum protein were calculated. The calculated results are shown in FIG. 1. In addition, FIG. 2 is a chart showing the stratum corneum sorption amounts per stratum corneum protein in all of layers 1 to 6 of the stratum corneum for the composition samples of the Examples and Comparative Example.

As is clear from the results in FIG. 1 and FIG. 2, it was found that the stratum corneum sorption amounts of the ultraviolet absorbers of Example 1 comprising Polymer 1 and Example 2 comprising Polymer 2 were both low compared to that of Comparative Example 1 not comprising the polymer of the present invention.

In other words, it was found that percutaneous penetration of octyl methoxycinnamate (log P value: 5.7) as an ultraviolet absorber was inhibited in Example 1 and Example 2 by comprising Polymer 1 and Polymer 2, respectively. From this result, it was suggested that the polymer of the present invention can be used as a percutaneous penetration inhibitor of octyl methoxycinnamate (log P value: 5.7) as an ultraviolet absorber.

### Example 3 and Comparative Example 2

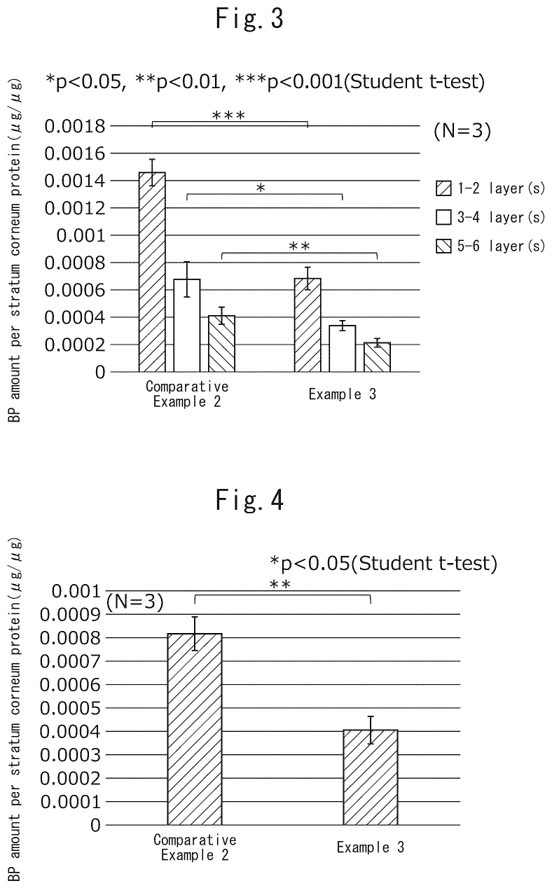

Based on the formulations in Table 3, cosmetic compositions of Example 3 and Comparative Example 2 were prepared. Each of the prepared compositions was subjected to the “human stratum corneum sorption test” described above. Results are shown in FIG. 3 and FIG. 4. Note that FIG. 3 is a chart showing results in which the stratum corneum sorption amounts per two layer portions of stratum corneum protein were each calculated, and FIG. 4 is a chart showing the stratum corneum sorption amount per stratum corneum protein in all of layers 1 to 6 of the stratum corneum.

TABLE 3
(Table 3 Example 3 and Comparative Example 2)

| | | | Comparative |
|---|---|---|---|
| Component | | Example 3 | Example 2 |
| Percutaneous | Polymer 1 | 5.0 | — |
| penetration inhibitor | | | |
| Preservative | Butylparaben | 0.05 | 0.05 |
| | (logP value: 3.6) | | |
| Additional | Ethanol | 10 | 10 |
| component | Ion-exchanged water | The balance | The balance |

| | | |
|---|---|---|
| Total | 100 | 100 |

Note that numerical values in Table 3 refer to blending amounts of components, and are in units of % by mass.

As is clear from the results of FIG. 3 and FIG. 4, it was found that the stratum corneum sorption amount of the preservative (butylparaben) of Example 3 comprising Polymer 1 was low compared to that of Comparative Example 2 not comprising the polymer of the present invention.

In other words, it was found that percutaneous penetration of butylparaben (log P value: 3.6) as a preservative was inhibited in Example 3 by comprising Polymer 1. From this result, it was suggested that the polymer of the present invention can be used as a percutaneous penetration inhibitor of butylparaben (log P value: 3.6) as a preservative.

### Example 4 and Comparative Example 3

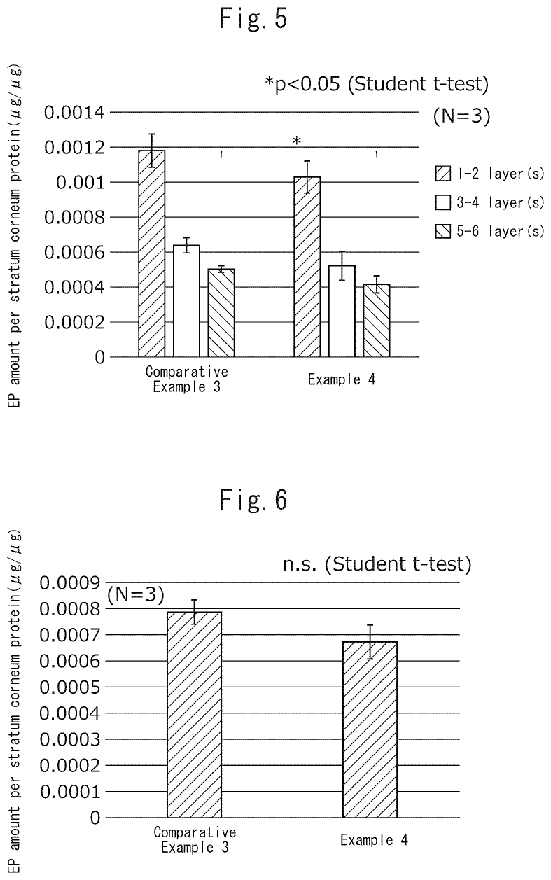

Based on the formulations in Table 4, cosmetic compositions of Example 4 and Comparative Example 3 were prepared. Each of the prepared compositions was subjected to the “human stratum corneum sorption test” described above. Results are shown in FIG. 5 and FIG. 6. Note that FIG. 5 is a chart showing results in which the stratum corneum sorption amounts per two layer portions of stratum corneum protein were each calculated, and FIG. 6 is a chart showing the stratum corneum sorption amount per stratum corneum protein in all of layers 1 to 6 of the stratum corneum.

TABLE 4
(Table 4 Example 4 and Comparative Example 3)

| | | | Comparative |
|---|---|---|---|
| Component | | Example 4 | Example 3 |
| Percutaneous | Polymer 1 | 5.0 | — |
| penetration inhibitor | | | |
| Preservative | Ethylparaben | 0.1 | 0.1 |
| | (logP value: 2.5) | | |
| Additional | Ion-exchanged water | The balance | The balance |
| component | | | |

| | | |
|---|---|---|
| Total | 100 | 100 |

Note that numerical values in Table 4 refer to blending amounts of components, and are in units of % by mass.

As is clear from the results of FIG. 5 and FIG. 6, it was found that the stratum corneum sorption amount of the preservative (ethylparaben) of Example 4 comprising Polymer 1 was low compared to that of Comparative Example 3 not comprising the polymer of the present invention.

In other words, it was found that percutaneous penetration of ethylparaben (log P value: 2.5) as a preservative was inhibited in Example 4 by comprising Polymer 1. From this result, it was suggested that the polymer of the present invention can be used as a percutaneous penetration inhibitor of ethylparaben (log P value: 2.5) as a preservative.

1. A composition that is a cosmetic composition comprising a polymer having a structure represented by formula (1) below and an oily component, 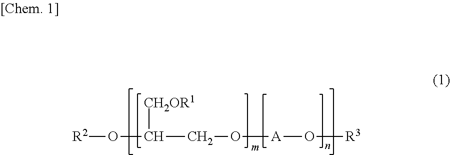
where in the formula (1), R¹, R², and R⁵ are each independently a hydrogen atom and an alkyl group having 1 to 4 carbon atoms; A is an alkylene group having 2 to 4 carbon atoms; and m and n are each independently 1.0 to 50, and
wherein a number average molecular weight of the polymer is 10,000 or less,
wherein an IOB value of the polymer is 0.4 to 1.8, and
wherein the oily component is an ultraviolet absorber and/or preservative having a log P value of 2.0 or greater.

2. The composition according to claim 1, wherein
in the formula (1), A is represented by formula (2) below: 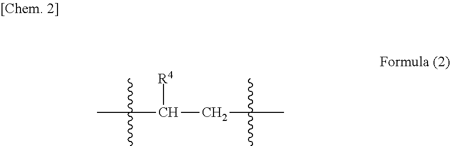
where in the formula (2), R⁴ is an alkyl group having 1 or 2 carbon atoms.

3. The composition according to claim 1, wherein the polymer is a random copolymer.

4. The composition according to claim 1, wherein in the formula (1), m is 2 or greater and at least a portion of R¹ includes a methyl group.

5. The composition according to claim 1, wherein in the formula (1), at least one of R² and R³ is a methyl group.

6. The composition according to claim 1, wherein the oily component has a log P value of 15.0 or less.

7. The composition according to claim 1, wherein the polymer is a percutaneous penetration inhibitor of the oily component.

8. A method of inhibiting percutaneous penetration of an oily component comprising contacting the oily component with a polymer having a structure represented by formula (1) below, that is an ultraviolet absorber and/or preservative having a log P value of 2.0 or greater: 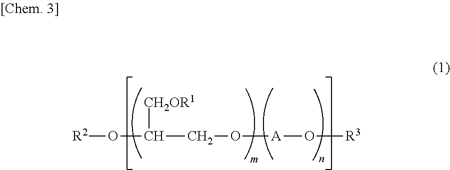
where in the formula (1), R¹, R², and R³ are each independently a hydrogen atom and an alkyl group having 1 to 4 carbon atoms; A is an alkylene group having 2 to 4 carbon atoms; and m and n are each independently 1.0 to 50, and wherein
a number average molecular weight of the polymer is 10,000 or less, and
an IOB value of the polymer is 0.4 to 1.8

9. The method according to claim 8, wherein
in the formula (1), A is represented by formula (2) below: 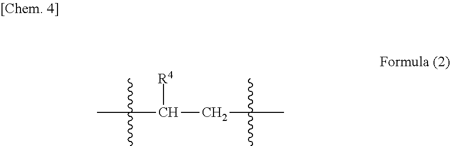
where in the formula (2), R⁴ is an alkyl group having 1 or 2 carbon atoms.

10. The method according to claim 9, wherein the polymer is a random copolymer.

11. The method according to claim 9, wherein in the formula (1), m is 2 or greater and at least a portion of R¹ includes a methyl group.

12. The method according to claim 9, wherein in the formula (1), at least one of R² and R³ is a methyl group.

13. The method according to claim 9, wherein the oily component has a log P value of 15.0 or less.
